(19) United States
(12) Patent Application Publication
(10) Pub. No.: US 2024/0122891 A1
(43) Pub. Date: Apr. 18, 2024

(54) COMPOSITIONS WITH ANTIMICROBIAL PROPERTIES

(71) Applicant: SYMRISE AG, Holzminden (DE)

(72) Inventors: Ravikumar PILLAI, Mahwah, NJ (US); Lars JUNG, Hamburg (DE); Martina KÖNIG, Hamburg (DE)

(21) Appl. No.: 17/768,375

(22) PCT Filed: Nov. 6, 2019
(86) PCT No.: PCT/EP2019/080324

§ 371 Date: Apr. 12, 2022

Related U.S. Application Data
(60) Provisional application No. 62/915,742, filed on Oct. 16, 2019.

(51) Int. Cl. A61K 31/23; A61K 8/36; A61Q 17/00

(52) U.S. Cl. CPC A61K 31/23; A61K 8/361; A61Q 17/005; A61K 2800/74

(57) ABSTRACT

Suggested is a cosmetic or pharmaceutical composition comprising (a) at least one alkanediol ester of carboxylic acids as antimicrobial agent, wherein the carboxylic acids contain 6 to 12 C atoms; (b) at least one emulsifier; (c) water; (d) and at least one antimicrobial agent.

### AREA OF INVENTION

The present invention belongs to the area of cosmetics and pharmaceuticals and belongs to novel compositions with antimicrobial properties.

### BACKGROUND OF THE INVENTION

In the cosmetics and pharmaceutical industry there is a constant need to use agents having antimicrobial properties in order to avoid microbiological spoilage of water containing preparations.

However, commercially available antimicrobial active agents may cause problems on the skin when used in higher concentrations. Additionally, those agents can exhibit undesirable side effects on the skin feel or the stability of the formulation.

Compositions containing antimicrobial agents are for example described in EP 2 589 291 A1 or in EP 2 774 604 A1. However, it is eligible to keep the dosage of such agents in the formulation as low as possible.

Therefore, it has been the object of the present invention to provide compositions that allow to minimize the necessary dosage of antimicrobial agents while keeping the antimicrobial protection of the total composition. Furthermore, it has been the object of the present invention to provide compositions which are toxicologically harmless, well tolerated by the skin, stable (in particular in the customary cosmetic and/or pharmaceutical formulations) and can be used in a low dosage.

### DESCRIPTION OF THE INVENTION

This object has been solved by the claims of the present invention. A first object of the present invention refers to a cosmetic or pharmaceutical composition comprising
(a) at least one alkanediol ester of carboxylic acids,
   wherein the carboxylic acids contain 6 to 12 C atoms;
(b) at least one emulsifier;
(c) water;
(d) and at least one antimicrobial agent.

The inventors have surprisingly found that at least one alkanediol ester of carboxylic acids, wherein the carboxylic acids contain 6 to 12 C atoms, improves the antimicrobial efficacy of commercially available antimicrobial agents.

According to the invention, a “antimicrobial agent”, which is component (d), refers to any antimicrobial agent which is commercially available.

Suitable anti-microbial agents are, in principle, all substances effective against bacteria, such as, for example, 4-hydroxybenzoic acid and its salts and esters, N-(4-chlorophenyl)-N′-(3,4-dichlorophenyl)urea, 2,4,4′-trichloro-2′-hydroxy-diphenyl ether (triclosan), 4-chloro-3,5-dimethyl-phenol, 2,2′-methylenebis(6-bromo-4-chlorophenol), 3-methyl-4-(1-methylethyl)phenol, 2-benzyl-4-chloro-phenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, oil of cloves, menthol, mint oil, farnesol, phenoxyethanol, benzyl alcohol, o-cymen-5-ol, chlorophenesin, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide, organic acids like benzoic acid, sorbic acid, salicylic acid or dehydroacetic acid, ethylhexyl glycerin, aprylhydroxamic acid or sorbitan caprylate. In a preferred embodiment according to the invention, the at least one further antimicrobial agent is selected from the group consisting of 1,2-alkanediol, in particular caprylyl glycol, hydroxyacetophenone, phenoxyethanol, benzyl alcohol, o-cymen-5-ol, chlorphenesin, benzoic acid, sorbic acid, salicylic acid and dehydroacetic acid.

In a preferred embodiment according to the invention the antimicrobial agent is selected from the group consisting of 1,2-alkanediol, in particular caprylyl glycol, hydroxyacetophenone, phenoxyethanol, benzyl alcohol, o-cymen-5-ol, chlorphenesin, benzoic acid, sorbic acid, salicylic acid and dehydroacetic acid.

In a preferred embodiment according to the invention, the alkanediol ester of carboxylic acids is an alkanediol diester. In a further preferred embodiment according to the invention, the alkanediol diester is an 1,3 propanediol diester. In a further preferred embodiment according to the invention, the carboxylic acid is selected from octanoic acid or decanoic acid. In a further preferred embodiment of the invention, the carboxylic acid is selected from a mixture of octanoic acid and decanoic acid. The weight percent ratio of the mixture of octanoic acid and decanoic acid can in one embodiment be in the range of from 10:90 to 90:10. In a more preferred embodiment the weight ratio of the mixture is in a range of from 30:70 to 70:30, further preferred from 40:60 to 60:40. In a most preferred embodiment according to the invention the weight ratio of the mixture is 60:40. In a most preferred embodiment according to the invention, the at least one alkanediol ester of carboxylic acids, wherein the carboxylic acids contain 6 to 12 C atoms, is an 1,3 propanediol diester of octanoic acid or decanoic acid or a mixture of octanoic and decanoic acid.

In one preferred embodiment according to the invention, component (a) is present in amount of from 1 to 10% by weight—calculated on the final composition. In one further preferred embodiment according to the invention the composition comprises the components in the following amount:
(a) 1 to 10% by weight of alkanediol esters of carboxylic acids containing 6 to 12 C atoms;
(b) 0.1 to 10% by weight of emulsifiers;
(c) 50 to 90% by weight of water;
(d) and 0.1 to 5% by weight of antimicrobial agents.

Emulsifiers

The compositions according to the present invention also include emulsifiers (component (b)). The emulsifiers may be of non-ionic, anionic, cationic and/or amphoteric nature.

In particular preferred are non-ionic emulsifiers, such as:
products of the addition of 2 to 30 mol ethylene oxide and/or 0 to 5 mol propylene oxide onto linear C8-22 fatty alcohols, onto C12-22 fatty acids and onto alkyl phenols containing 8 to 15 carbon atoms in the alkyl group;
C12/18 fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C6/22 fatty acids, ricinoleic acid and 12-hydroxystearic acid and glycerol, polyglycerol, pentaerythritol, -dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, di- and/or tri-PEG-alkyl phosphates and salts thereof;
wool wax alcohols;
Lecithin and hydrolysed lecithin;
polysiloxane/polyalkyl polyether copolymers and corresponding derivatives;
mixed esters of pentaerythritol, fatty acids, citric acid and fatty alcohol and/or mixed esters of C6-22 fatty acids, methyl glucose and polyols, preferably glycerol or polyglycerol,
polyalkylene glycols and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C12/18 fatty acid monoesters and diesters of addition products of ethylene oxide onto glycerol are known as lipid layer enhancers for cosmetic formulations. The most preferred emulsifiers are described in more detail as follows:

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesqui-hydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are aliphatic C12-22 fatty acids, such as palmitic acid, stearic acid or behenic acid for example, and C12-22 dicarboxylic acids, such as azelaic acid or sebacic acid for example. Further typical anionic emulsifiers are Mono-, Di- and Trialkyl phosphates and Mono-, di- and/or Tri-PEG-Alkyl Phosphates and salts thereof, such as Cetyl Phosphate Potassium salt or Citrate ester such as Glycerol Oleate Citrate and Glycerol Stearate Citrate.

Amphotheric or Zwitterionic Emulsifiers

Other suitable emulsifiers are amphoteric or zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C8/18 alkyl or acyl group, contain at least one free amino group and at least one —COOH— or —SO3H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylamino-acetic acids containing around 8 to 18 carbon atoms in the alkyl group. Particularly preferred ampholytic surfactants are N-cocoalkylaminopropionate, cocoacylaminoethyl aminopropionate, C12/18 acyl sarcosine Sodium Lauryl Sulfoacetate and Sodium Oleoyl Sarcosinate.

In a preferred embodiment according to the invention, the emulsifiers are selected from the group consisting of non-ionic, anionic, cationic, or amphoteric emulsifiers and their mixtures. In a further preferred embodiment according to the invention the emulsifier is selected from Sodium Lauryl Sulfoacetate and Sodium Oleoyl Sarcosinate.

Of course the compositions according to the invention may contain further ingredients which are in particular suitable for use in cosmetic or pharmaceutical compositions, for example thickening agents and rheology additives, polymers, sun protection factors, actives modulating skin and/or hair pigmentation, anti-ageing actives, hair growth activators or inhibitors, cooling agents, odour absorbers and antiperspirant active agents, carriers and hydrotropes, perfume oils and fragrances and/or dyes. These ingredients are well known in the state of the art.

Compositions according to the present invention are cosmetic or pharmaceutical compositions. For example, the may be selected from the group of products for treatment, protecting, care and cleansing of the skin and/or hair or as a make-up product.

The compositions according to the invention are preferably in the form of an emulsion, e.g. W/O (water-in-oil), O/W (oil-in-water), W/O/W (water-in-oil-in-water), O/W/O (oil-in-water-in-oil) emulsion, PIT emulsion, Pickering emulsion, emulsion with a low oil content, micro- or nanoemulsion, a solution, e.g. in oil (fatty oils or fatty acid esters, in particular C6-C32 fatty acid C2-C30 esters) or silicone oil, dispersion, suspension, creme, lotion or milk, depending on the production method and ingredients, a gel (including hydrogel, hydrodispersion gel, oleogel), spray (e.g. pump spray or spray with propellant) or a foam or an impregnating solution for cosmetic wipes, a detergent, e.g. soap, synthetic detergent, liquid washing, shower and bath preparation, bath product (capsule, oil, tablet, salt, bath salt, soap, etc.), effervescent preparation, a skin care product such as e.g. an emulsion (as described above), ointment, paste, gel (as described above), oil, balsam, serum, powder (e.g. face powder, body powder), a mask, a pencil, stick, roll-on, pump, aerosol (foaming, non-foaming or post-foaming), a deodorant and/or antiperspirant, mouthwash and mouth rinse, a foot care product (including keratolytic, deodorant), an insect repellent, a sunscreen, aftersun preparation, a shaving product, aftershave balm, pre- and aftershave lotion, a depilatory agent, a hair care product such as e.g. shampoo (including 2-in-1 shampoo, anti-dandruff shampoo, baby shampoo, shampoo for dry scalps, concentrated shampoo), conditioner, hair tonic, hair water, hair rinse, styling creme, pomade, perm and setting lotion, hair spray, styling aid (e.g. gel or wax), hair smoothing agent (detangling agent, relaxer), hair dye such as e.g. temporary direct-dyeing hair dye, semi-permanent hair dye, permanent hair dye, hair conditioner, hair mousse, eye care product, make-up, make-up remover or baby product.

The compositions according to the invention are particularly preferably in the form of an emulsion, in particular in the form of a W/O, O/W, W/O/W, O/W/O emulsion, PIT emulsion, Pickering emulsion, emulsion with a low oil content, micro- or nanoemulsion, a gel (including hydrogel, hydrodispersion gel, oleogel), a solution e.g. in oil (fatty oils or fatty acid esters, in particular C6-C32 fatty acid C2-C30 esters)) or silicone oil, or a spray (e.g. pump spray or spray with propellant).

Auxiliary substances and additives can be included in quantities of 5 to 99% b.w., preferably 10 to 80% b.w., based on the total weight of the composition. The amounts of cosmetic or dermatological auxiliary agents and additives and perfume to be used in each case can easily be determined by the person skilled in the art by simple trial and error, depending on the nature of the particular product.

In a preferred embodiment according to the invention, the compositions with antimicrobial properties according to the invention are effective against all microorganisms which may be present in cosmetic or pharmaceutical compositions. In a more preferred embodiment according to the invention the microorganisms are selected from the group consisting of Escherichia coli, Pseudomonas aeruginosa, Staphylococcus aureus, Candidatus albicans and Aspergillus brasiliensis. In a most preferred embodiment according to the invention the microorganisms are Candidatus albicans and Aspergillus brasiliensis.

A further embodiment of the present invention refers to the use of at least one alkanediol ester of carboxylic acids containing 6 to 12 C atoms as an antimicrobial efficacy enhancer in cosmetic or pharmaceutical compositions.

In a preferred embodiment according to the invention the at least one alkanediol ester is an alkanediol diester. In a further preferred embodiment according to the invention the alkanediol diester is an 1,3 propanediol diester. In a further preferred embodiment according to the invention the carboxylic acid is selected from octanoic acid or decanoic acid. In a most preferred embodiment according to the invention, the at least one alkanediol ester of carboxylic acids, wherein the carboxylic acids contain 6 to 12 C atoms, is an 1,3 propanediol diester of octanoic acid or decanoic acid.

Furthermore, as mentioned above, the at least one alkanediol ester of carboxylic acids containing 6 to 12 C atoms improves the antimicrobial efficacy of antimicrobial agents. Suitable antimicrobial agents according to the invention have been already listed above.

A further embodiment of the invention relates to a method for improving the antimicrobial efficacy of compositions comprising further antimicrobial agents comprising the following steps:
(a) Providing an oil phase comprising an emulsifier and at least one antimicrobial agent;
(b) Contacting the oil phase with at least one alkanediol ester of carboxylic acids containing 6 to 12 C atoms; and
(c) Mixing the oil phase from step b containing the at least one alkanediol ester of carboxylic acids containing 6 to 12 C atoms with an aqueous phase.

In a preferred embodiment according to the invention the compositions are selected from cosmetic or pharmaceutical compositions. Of course, all ingredients mentioned above and/or known in the state of the art which are suitable for cosmetic and/or pharmaceutical compositions can also be used in the method according to the invention. Furthermore, the amounts of the additional ingredients can in each case easily be determined by the person skilled in the art by simple trial and error, depending on the nature of the particular product.

In a preferred embodiment according to the invention the at least one alkanediol ester is an alkanediol diester. In a further preferred embodiment according to the invention the alkanediol diester is an 1,3 propanediol diester. In a further preferred embodiment according to the invention the carboxylic acid is selected from octanoic acid or decanoic acid. In a further preferred embodiment according to the invention the carboxylic acid is selected from octanoic acid or decanoic acid. In a further preferred embodiment of the invention, the carboxylic acid is selected from a mixture of octanoic acid and decanoic acid. The weight percent ratio of the mixture of octanoic acid and decanoic acid can in one embodiment be in the range of from 10:90 to 90:10. In a more preferred embodiment the weight ratio of the mixture is in a range of from 30:70 to 70:30, further preferred from 40:60 to 60:40. In a most preferred embodiment according to the invention the weight ratio of the mixture is 60:40. In a most preferred embodiment according to the invention, the at least one alkanediol ester of carboxylic acids, wherein the carboxylic acids contain 6 to 12 C atoms, is an 1,3 propanediol diester of octanoic acid or decanoic acid or or a mixture of octanoic and decanoic acid.

### EXAMPLES

### Example 1

### Preparation of Compositions According to the Invention

Table 1 shows the ingredients of compositions A to C, wherein compositions B and C are according to the invention and composition A serves for comparison purposes.

The ingredients of phase A have been premixed without the thickener and were then heated to approximately 80° C. After that, the thickener was added and the mixture was dispersed by using an Ultra-Turrax® (3500 rpm, 15 sec/100 g).

All ingredients of phase B have been mixed and were then heated to approximately 80° C. After that, phase B was added to phase A, and the homogenizing process was started by Ultra-Turrax® (10000 rpm, 1 min/100 g).

TABLE 1
Ingredients of the compositions A, B and C

| | | | A | B | C |
|---|---|---|---|---|---|
| Phase | Ingredients | INCI | % w/w | % w/w | % w/w |
| A | Emulsiphos ® | POTASSIUM CETYL PHOSPHATE, | 1.50 | 1.50 | 1.50 |
| | (677660) | HYDROGENATED PALM | | | |
| | | GLYCERIDES | | | |
| | SymMollient ® PDCC | PROPANEDIOL | 3.00 | 3.00 | 3.00 |
| | | DICAPRYLATE/CAPRATE | | | |
| | Paraffin Oil | PARAFFIN OIL | 7.00 | 7.00 | 7.00 |
| | Carbopol EDT 2050 | CARBOMER | 0.50 | 0.50 | 0.50 |
| B | Wasser | AQUA | 86.90 | 86.50 | 86.50 |
| | SymSave ® H | HYDROXYACETOPHENONE | — | 0.40 | — |
| | Hydrolite ® CG | CAPRYLYL GLYCOL | — | — | 0.40 |
| | Sodium Hydroxide | AQUA, SODIUM HYDROXIDE | 1.10 | 1.10 | 1.10 |
| | 10% ig | | | | |
| | Total amount | | 100.00 | 100.00 | 100.00 |

Table 2 shows the results of the antimicrobial enhancement effect of propanediol dicaprylate/caprate. It can be seen that composition C, containing propanediol dicaprylate/caprate and caprylyl glycol, wherein caprylyl glycol is an antimicrobial agent, shows the best antimicrobial efficacy, in particular for samples which were inoculated with EC, CA and AN. Compared to this, composition A, which contains no antimicrobial agent, shows the worst results.

TABLE 2
Animicrobial efficacy of compositions A, B and C

| Sam- | | Inoculum | | | | |
|---|---|---|---|---|---|---|
| ple | | [×10E6] | 2 days | 7 days | 14 days | 28 days |
| A | EC | 4100000 | 1600000 | 2400000 | 750000 | 3600000 |
| | PS | 4500000 | 2400000 | 3000000 | 1400000 | 2500000 |
| | SA | 4600000 | 36000 | 150 | 100 | <10 |
| | CA | 5100000 | 2400000 | 630000 | 53000 | 450000 |
| | AN | 4800000 | 7000000 | 13000000 | 3900000 | 7000000 |
| B | EC | 4100000 | 30 | <10 | <10 | <10 |
| | PS | 4500000 | <10 | <10 | <10 | <10 |
| | SA | 4600000 | <10 | <10 | <10 | <10 |
| | CA | 5100000 | 3200000 | 3700 | <10 | <10 |
| | AN | 4800000 | 490000 | 23000 | 170 | <10 |
| C | EC | 4100000 | <10 | <10 | <10 | <10 |
| | PS | 4500000 | <10 | <10 | <10 | <10 |
| | SA | 4600000 | <10 | <10 | <10 | <10 |
| | CA | 5100000 | 600000 | <10 | <10 | <10 |
| | AN | 4800000 | 600000 | <10 | <10 | <10 |

Legend: EC = Escherichia coli; PS = Pseudomonas aeruginosa; SA = Staphylococcus aureus; CA = Candidatus albicans; AN = Aspergillus brasiliensis

Table 3 shows the ingredients of compositions H and K, wherein composition H is according to the invention and composition K serves for comparison purposes.

The ingredients of phase A have been premixed without the thickener and were then heated to approximately 80° C. After that, the thickener was added and the mixture was dispersed by using an Ultra-Turrax® (3500 rpm, 15 sec/100 g).

All ingredients of phase B have been mixed and were then heated to approximately 80° C. After that, phase B was added to phase A, and the homogenizing process was started by Ultra-Turrax® (10000 rpm, 1 min/100 g).

TABLE 3
Ingredients of the compositions H and K

| | | | H | K |
|---|---|---|---|---|
| | | | % | % |
| Phase | Ingredients | INCI | w/w | w/w |
| A | Emulsiphos ® | POTASSIUM CETYL | 1.50 | 1.50 |
| | (677660) | PHOSPHATE, | | |
| | | HYDROGENATED | | |
| | | PALM GLYCERIDES | | |
| | SymMollient ® | PROPANEDIOL | 3.00 | — |
| | PDCC | DICAPRYLATE/ | | |
| | | CAPRATE | | |
| | Paraffin Oil | PARAFFIN OIL | 7.00 | 10.00 |
| | Carbopol ® EDT | CARBOMER | 0.50 | 0.50 |
| | 2050 | | | |
| B | Wasser | AQUA | 86.40 | 86.40 |
| | Hydrolite ® 6 | 1,2-HEXANEDIOL | 0.50 | 0.50 |
| | Sodium | AQUA, SODIUM | 1.10 | 1.10 |
| | Hydroxide | HYDROXIDE | | |
| | 10% ig | | | |
| | Total amount | | 100.00 | 100.00 |

Table 4 shows the results of the antimicrobial enhancement effect of propanediol dicaprylate/caprate. It can be seen that composition H, containing propanediol dicaprylate/caprate and 1,2 hexanediol, wherein 1,2 hexanediol is an antimicrobial agent, shows the best antimicrobial efficacy. Compared to this, composition K, which does not contain propanediol dicaprylate/caprate, shows worse results.

TABLE 4
Animicrobial efficacy of compositions H and K

| Sam- | | Inoculum | | | | |
|---|---|---|---|---|---|---|
| ple | | [×10E6] | 2 days | 7 days | 14 days | 28 days |
| H | CA | 6.0 × E6 | 1.1 × E6 | 7.5 × E5 | 4.0 × E5 | 1.1 × E5 |
| | AN | 4.8 × E6 | 1.7 × E7 | 1.3 × E7 | 4.3 × E6 | 3.5 × E6 |
| K | CA | 6.0 × E6 | 3.1 × E6 | 2.2 × E6 | 1.4 × E6 | 5.5 × E5 |
| | AN | 4.8 × E6 | 1.6 × E7 | 6.0 × E6 | 1.0 × E7 | 8.5 × E6 |

Legend: CA = Candidatus albicans; AN = Aspergillus brasiliensis

1. A cosmetic or pharmaceutical composition comprising
(a) at least one alkanediol ester of carboxylic acids, wherein the carboxylic acids contain 6 to 12 C atoms;
(b) at least one emulsifier;
(c) water;
(d) and at least one antimicrobial agent.

2. The composition according to claim 1, wherein the at least one antimicrobial agent is selected from the group consisting of 1,2-alkanediol, in particular caprylyl glycol, hydroxyacetophenone, phenoxyethanol, benzyl alcohol, o-cymen-5-ol, chlorphenesin, benzoic acid, sorbic acid, salicylic acid and dehydroacetic acid, and mixtures thereof.

3. The composition according to claim 1, wherein the at least one alkanediol ester is an alkanediol diester.

4. The composition according to claim 3, wherein the alkanediol diester is an 1,3 propanediol diester.

5. The composition according to claim 1, wherein the carboxylic acid is selected from octanoic acid or decanoic acid or mixtures of octanoic acid and decanoic acid.

6. The composition according to claim 1, wherein component (a) is present in amount of from 1 to 10% by weight—calculated on the final composition.

7. The composition according to claim 1, wherein the emulsifiers are selected from the group consisting of non-ionic, anionic, cationic, or amphoteric emulsifiers, and mixtures thereof.

8. The composition according to claim 1, wherein the composition comprises the components in the following amount:
(a) 1 to 10% by weight of alkanediol esters of carboxylic acids containing 6 to 12 C atoms;
(b) 0.1 to 10% by weight of emulsifiers;
(c) 50 to 90% by weight of water; and
(d) and 0.1 to 5% by weight of antimicrobial agents.

9. A method of enhancing antimicrobial efficacy in cosmetic or pharmaceutical compositions, comprising adding at least one alkanediol ester of carboxylic acids containing 6 to 12 C atoms.

10. The method according to claim 9, wherein the at least one alkanediol ester is an alkanediol diester.

11. The method according to claim 9, wherein the alkanediol diester is 1,3 propanediol diester.

12. The method according to claim 9, wherein the carboxylic acid is selected from octanoic acid or decanoic acid or mixtures of octanoic acid and decanoic acid.

13. The method according to claim 9, wherein the at least one alkanediol ester of carboxylic acids containing 6 to 12 C atoms also improves the antimicrobial efficacy of antimicrobial agents.

14. A method for improving the antimicrobial efficacy of compositions comprising antimicrobial agents, comprising the following steps:
(a) Providing an oil phase comprising an emulsifier and at least one antimicrobial agent;
(b) Contacting the oil phase with at least one alkanediol ester of carboxylic acids containing 6 to 12 C atoms; and
(c) Mixing the oil phase from step (b) containing the at least one alkanediol ester of carboxylic acids containing 6 to 12 C atoms with an aqueous phase.

15. The method according to claim 14, wherein the at least one alkanediol ester of carboxylic acids containing 6 to 12 C atoms is 1,3 propanediol diester of octanoic or decanoic acids.
